(19) United States
(12) Patent Application Publication
(10) Pub. No.: US 2025/0093338 A1
(43) Pub. Date: Mar. 20, 2025

(54) METHODS AND SYSTEMS FOR RAPID DETECTION OF ANALYTES

(71) Applicant: University of Georgia Research Foundation, Inc., Athens, GA (US)

(72) Inventors: Yiping Zhao, Bogart, GA (US); Ralph A. Tripp, Watkinsville, GA (US); Yanjun Yang, Athens, GA (US); XianYan Chen, Bogart, GA (US); Hemant K. Naikare, Tifton, GA (US)

(21) Appl. No.: 18/500,860

(22) Filed: Nov. 2, 2023

Related U.S. Application Data
(60) Provisional application No. 63/582,623, filed on Sep. 14, 2023.

(51) Int. Cl. G01N 33/53; G01N 21/65; G01N 33/569; G06N 3/0442; G06N 3/045; G06N 3/0464

(52) U.S. Cl. CPC G01N 33/5308; G01N 21/658; G01N 33/56983; G06N 3/0442; G06N 3/045; G06N 3/0464

(57) ABSTRACT

The present disclosure provides for surface-enhanced Raman spectroscopy (SERS) systems and methods for detecting, analyzing, and/or quantifying biomolecules or biological agents using SERS systems and a neural network model. The biological agent can be a virus, such as a coronavirus (e.g., SARS-CoV-2 or a variant thereof).

### CROSS REFERENCE TO RELATED APPLICATIONS

This application claims the benefit of U.S. Provisional Application No. 63/582,623, entitled “METHODS AND SYSTEMS FOR RAPID DETECTION OF SARS-COV-2” and filed on Sep. 14, 2023, which is incorporated herein by reference in its entirety.

### BACKGROUND

Detecting viruses, such as corona virus, in humans is of great importance. Detecting the virus in humans with a high degree of accuracy for positive and negative, detecting quickly, and at a desired cost point is desired. Thus, there is a need in the industry to achieve these goals.

### SUMMARY

The present disclosure provides for surface-enhanced Raman spectroscopy (SERS) systems and methods for detecting, analyzing, and/or quantifying biomolecules or biological agents using SERS systems and a neural network (e.g., a recurrent neural network (RNN)) model.

In an aspect, the present disclosure provides for a method for detecting the presence of a biological agent comprising: disposing a sample onto a surface enhanced Raman spectroscopy (SERS) detecting module, wherein the SERS detecting module comprises a substrate having an array of nanorods on a surface of the substrate, wherein the tilt angle (s) between an individual nanorod and the surface is about 0° to about 90°; measuring at least one SERS spectrum; and providing the SERS spectrum to a first neural network (e.g., recurrent neural network (RNN) model) trained to detect the presence or absence of a biological agent in the sample. When the biological agent is present in the sample, the method includes providing the SERS spectrum to a second RNN model trained to quantify the amount of biological agent present.

The present disclosure also provides for a system for detecting the presence of a biological agent comprising: a SERS detecting module having the characteristic of being able to receive a sample, wherein the SERS detecting module comprises a substrate having an array of nanorods on a surface of the substrate, wherein the tilt angle (p) between an individual nanorod and the surface is about 0° to about 90°; a light source that is directed towards the substrate; a SERS detection system to measure at least one surface enhanced Raman spectroscopy (SERS) spectrum; and an analysis system configured to receive the SERS spectrum, wherein the analysis system includes a first recurrent neural network (RNN) model trained to detect the presence or absence of a biological agent in the sample and a second RNN model trained to quantify the amount of biological agent present.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates direct quantitative detection from human nasopharyngeal swabs (HNS) specimens using surface-enhanced Raman spectroscopy (SERS) and deep learning algorithms (DLAs).

FIG. 2A illustrates an averaged SERS spectra for individual HNS specimens: positive, negative, and inactivation buffer (not averaged).

FIG. 2B illustrates SERS peak alignments of different reagents in inactivation buffer.

FIGS. 2C-2D illustrate a principal component analysis (PCA) score plot (FIG. 2C) and a t-distributed stochastic neighbor embedding (tSNE) plot (FIG. 2D) of clustering results based on SERS spectra of HNS specimens and inactivation buffer.

FIG. 3A illustrates an architecture of the recurrent neural network (RNN) model.

FIG. 3B illustrates loss and accuracy curves for the test spectral set.

FIGS. 3C-3D illustrate a confusion matrix (FIG. 3C) and a corresponding receiver operating characteristic (ROC) curve (FIG. 3D) of the RNN classification results of the test spectral set.

FIG. 3E illustrates a summary of the training and testing accuracies of different machine-learning algorithms (MLA) and DLA models for SERS spectrum classification.

FIG. 4A illustrates a feature importance map (FIM) of the RNN model to differentiate negative and positive spectra. The black dotted line marks the threshold of 50%, and the purple segmented lines under the FIM indicate the R_{RNN}.

FIG. 4B illustrates an obtained R_{K} from Raman spectra of lipid, amide I, amide III, RNA, tyrosine, and phenylalanine and corresponding matching scores.

FIG. 5A illustrates an architecture of the RNN regression model.

FIG. 5B illustrates loss curves for the training spectral and test spectral sets.

FIG. 5C illustrates plots of R² and RMSE for different regression models.

FIG. 5D illustrates plots of a predicted cycle threshold (Ct) value (Ctₚᵣₑ) versus actual Ct values (Ct_{act}) of Orf1 ab (ORF1 ab) gene, nucleocapsid (N) gene, spike (S) gene for RNN, back-propagation (BP), and convolutional neural network (CNN) regression models.

FIG. 6A illustrates a plot of ratio γ based on the results from the RNN model against specimen number (arranged particular).

FIG. 6B illustrates plots of a predicted Ctₚᵣₑ versus actual Ct_{act} of ORF1 ab gene, N gene, S gene for the blind test. The dash line in each plot indicates Ct_{act}=Ctₚᵣₑ.

FIG. 7 illustrates a representative scanning electron microscopy (SEM) image of an AgNR@SiO₂ array.

FIGS. 8A-8D illustrate a typical raw SERS spectrum (FIG. 8A) and a baseline fitting curve (FIG. 8B) obtained from the raw spectrum. FIGS. 8C and 8D illustrate the baseline-corrected (FIG. 8C) and the baseline-corrected and normalized (FIG. 8D) spectrum.

FIG. 8E illustrates 20 SERS spectra from one positive specimen after pre-processing using a similar process to that illustrated in FIGS. 8A-8D.

FIG. 9 illustrates averaged SERS spectra from individual positive and negative HNS specimens as well as from an inactivation buffer.

FIG. 10 illustrates the comparison of an average SERS spectrum of a positive specimen, a negative specimen, and a buffer.

FIG. 11 illustrates an architecture of the BP model.

FIG. 12 illustrates an architecture of the CNN model.

FIG. 13 illustrates a flowchart for calculating feature importance.

FIG. 14 illustrates linear fit plots for N gene, S gene, and ORF1ab gene.

FIG. 15 illustrates Ct value regression results for ORF1 ab gene, N gene, and S gene using random forest regression and support vector regression. The x-axis is the measured Ct values from RT-PCR and the y-axis represents the predicted Ct values from different ML/DL models.

FIG. 16 illustrates an architecture of the BP regression model.

FIG. 17 illustrates an architecture of the CNN regression model.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

Embodiments of the present disclosure will employ, unless otherwise indicated, techniques of chemistry, mechanical engineering, bio-medical engineering, material science, and the like, which are within the skill of the art.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent. Prior to describing the various embodiments, the following definitions are provided and should be used unless otherwise indicated.

### Definitions

The term “biomolecule” or “biological agent” is intended to encompass deoxyribonucleic acid (DNA), ribonucleic acid (RNA), nucleotides, oligonucleotides, nucleosides, proteins, peptides, polypeptides, selenoproteins, antibodies, protein complexes, combinations thereof, and the like. In particular, the biomolecule or biological agent can include, but is not limited to, naturally occurring substances such as polypeptides, polynucleotides, lipids, fatty acids, glycoproteins, carbohydrates, fatty acids, fatty esters, macromolecular polypeptide complexes, vitamins, co-factors, whole cells, eukaryotic cells, prokaryotic cells, microorganisms such as viruses, bacteria, protozoa, archaea, fungi, algae, spores, apicomplexan, trematodes, nematodes, mycoplasma, or combinations thereof.

In a preferred aspect, the biomolecule or biological agent is a virus, including, but not limited to, RNA and DNA viruses. In particular, the biomolecule is a virus, which may include, but is not limited to, negative-sense and positive-sense RNA viruses and single stranded (ss) and double stranded (ds) DNA viruses. The ds group I DNA viruses include the following families: Adenoviridae, Herpesviridae, Papillomaviridae, Polyomaviridae, Poxyiridae, and Rudiviridae. The group II ssDNA viruses include the following families: Microviridae, Geminiviridae, Circoviridae, Nanoviridae, and Parvoviridae. The ds group Ill RNA viruses include the following families: Birnaviridae and Reoviridae. The group IV positive-sense ssRNA virus families: Arteriviridae, Coronaviridae, Astroviridae, Caliciviridae, Flaviviridae, Hepeviridae, Picornaviridae, Retroviridae and Togaviridae. The group V negative-sense ssRNA virus families: Bornaviridae, Filoviridae, Paramyxoviridae, Rhabdoviridae, Arenaviridae, Bunyaviridae, and Orthomyxoviridae.

The term “types” with reference to viruses is intended to include different families and/or genuses of viruses. Thus, for instance, the phrase “different types of viruses' refers to viruses from different genuses or different families (e.g., HIV and influenza) and does not refer to different strains of viruses of the same genus or family, such as different strains of HIV (e.g., Ball, LAV, and NL4-4) or influenza (e.g., influenza A and influenza B). It should also be noted that, as used herein, “different strains” may refer to different strains/species of virus and/or to different sub groups of viruses within the same strain, such as different influenza viruses of influenza A (e.g., HKX-31 (HN), A/WSN/33 (H1 N1), and A/PR/8234 (H1N1)).

The term “Surface-Enhanced Raman Scattering (SERS)” refers to the increase in Raman scattering exhibited by certain molecules in proximity to certain metal surfaces. The SERS effect can be enhanced through combination with the resonance Raman effect. The surface-enhanced Raman scattering effect is even more intense if the frequency of the excitation light is in resonance with a major absorption band of the molecule being illuminated. In short, a significant increase in the intensity of Raman light scattering can be observed when molecules are brought into close proximity to (but not necessarily in contact with) certain metal surfaces.

The term “detectable signal” is a SERS signal. The SERS signal is detectable and distinguishable from other background signals that are generated from sample. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the detectable signal and/or the background.

A neural network can include many parameters (tens of thousands, millions, or sometimes even billions or more) and can be trained on large quantities of unlabeled text using self-supervised learning or semi-supervised learning techniques. Some neural networks can be generative—that is they can generate new data based at least in part on patterns and structure learned from their input training data. Examples of neural networks include various versions of OPENAI's Generative Pre-trained Transformer (GPT) model (e.g., GPT-1, GPT-2, GPT-3, GPT-4, etc.), META's Large Language Model Meta AI (LLaMA), and GOOGLE's Pathways Language Model 2 (PaLM 2), among others. Neural networks can be configured to return a response to a prompt, which can be in a structured form (e.g., a request or prompt with a predefined schema and/or parameters) or in an unstructured form (e.g., free form or unstructured text). The present disclosure (systems and methods) can use convolutional neural networks and/or recurrent neural networks (RNN) to perform various tasks and purposes. The present disclosure (systems and methods) can use convolutional neural networks and RNN where each perform a different task or have a different purpose.

### Discussion

The present disclosure, in one aspect, provides for surface-enhanced Raman spectroscopy (SERS) systems and methods for detecting, analyzing, and/or quantifying biomolecules or biological agents using SERS systems and a neural network (e.g., a recurrent neural network (RNN)) model. In some aspects, the biological agent can be a virus, such as a coronavirus (e.g., SARS-CoV-2 or a variant thereof). The combination of SERS techniques with neural network (e.g., RNN) model analysis can provide accurate, rapid, and cost-effective detection of biological agents.

SERS can be used to as a diagnostic platform for detecting viruses such as SARS-CoV-2 due to its sensitivity, ability to provide unique spectral features for different viruses, inherent simplicity, and capability for use in a point-of-care detection device. The present disclosure provides for SERS systems and methods that provide SERS-based SARS-CoV-2 detection that is portable to currently used techniques. In an embodiment, the present disclosure provides for a SERS methods and systems that have the following three capabilities: the ability to achieve effective classification of specimens as either SARS-CoV-2 positive or negative, the ability to attain accurate quantification of the SARS-CoV-2 viral load, specifically the cycle threshold (Ct) value, and the ability to successfully detect the virus within genuine clinical specimens.

In an aspect, the SERS system for detecting the presence of a biological agent in a sample can include a SERS detecting module upon which a sample can be disposed. The SERS detecting module can include an array of nanostructures on the surface of the substrates. In some aspects, the nanostructure is a nanorod. The nanostructure (e.g., nanorods) can be fabricated from a metal, a metal oxide, a metal nitride, a metal oxynitride, a polymer, a multicomponent material, or a combination thereof. In a further aspect, the metal nanostructure (e.g., nanorods) can be silver, nickel, aluminum, silicon, gold, platinum, palladium, titanium, cobalt, copper, zinc, oxides of each, nitrides of each, oxynitrides of each, carbides of each, or a combination thereof. The SERS detecting module can include a nanorod array. The nanostructures (e.g., nanorods) can be further coated with SiO₂. In an aspect, the SERS system can be a SiO₂-coated silver nanorod array. For a nanorod array, the tilt angle between an individual nanorods and the surface of the substrate can range from about 0° to about 90°, about 10° to about 90°, 20° to about 80°, about 50° to about 80°, about 60° to about 80°, about 70° to about 90°, or about 77°.

In general, the SERS system can include a light source (e.g., a laser) or is adapted to direct a light source (e.g., uses lenses and/or fibers to guide the light) that may be generated separately from the SERS system, and a device or structure to receive or detect Raman scattered light energy (e.g., uses a fiber to collect light). Optionally the SERS system can include one or more lenses to guide the light and the scattered Raman light energy, one or more mirrors to direct the laser light or scattered Raman light energy, and/or one or more filters to select certain wavelengths of light and/or scattered Raman light energy. The resulting light (e.g., detectable SERS signal) can then be measured by a device (e.g., a spectrometer/CCD). In an embodiment, the SERS system can include collection and measurement devices or instruments to collect and measure the detectable scattered Raman light energy signal.

The SERS system can include multiple different neural networks that can used for different purposes such as those described below, in the Examples, and otherwise herein. The SERS system and method can use convolutional neural networks (CNN) and/or recurrent neural networks (RNN) to perform various tasks. While the following discussion references RNN, convolutional neural networks can also be used or RNN and convolutional neural networks can be used for different purposes. In an aspect, the SERS system can include multiple different RNN models can be used for different purposes, such as using a first RNN model to determine the presence of a biological agent and using a second RNN model to quantify the biological agent present. The first RNN model can include one or more sets of layers. In some aspects, these layers can include one or at least one convolutional layer; one or at least one pool layer; three consecutive blocks, where a single block comprises one convolutional block and two identity blocks; two or at least two recurrent layers; and one or at least one fully connected layer. In some aspects, the set of recurrent layers of the first RNN model can be two or at least two long short-term memory layers. The convolutional block can include a convolutional layer, a batch normalization step, a corrected linear transform step, and a pool layer. The identity block can include a convolutional layer, a batch normalization step, a corrected linear transform step, and a pool layer. A convolutional block and/or layers of recurrent cells (e.g., long short-term memory layers) can process sequential data to detect local patterns of the input data. An identity block and/or a “hidden state” can be updated periodically to maintain a residual mapping that can be used to better approximate how sequential data can be combined. The inclusion of an identity block can mitigate chances of the vanishing gradient problem occurring, which could prevent the RNN from further training when performing backpropagation. The second RNN model can include one or more sets of layers. In some aspects, these layers can include a set of two or more recurrent layers, a set of two or at least two dropout layers, and a set of three or at least three fully connected layers. In some aspects, the set of recurrent layers of the second RNN model can be two or at least two long short-term memory layers. Additional details are provided in the Example.

In an aspect, the SERS method for detecting the presence of a biological agent in a sample can include disposing the sample onto the SERS detecting module. In particular, the sample can be disposed on the array of nanostructures. The method includes detecting the presence of a biological agent in a sample by measuring at least one SERS spectrum of the substrate. The SERS spectrum is provided to a first recurrent neural network (RNN) model trained (as described below and herein) to detect the presence or absence of a biological agent in the sample. When the biological agent is confirmed to be present in the sample, the method can further include providing the SERS spectrum to a second RNN model trained to quantify (as described below and herein) the amount of biological agent present in the sample. The entire time to perform the method can take from about 1 to about 20 minutes, from about 1 to about 15 minutes, from about 5 to about 15 minutes, from about 10 to about 15 minutes, less than 15 minutes, or less than 10 minutes.

In regard to the deep learning for the neural networks (e.g., RNN model and/or CNN model), positive and negative specimens are gathered from numerous patients from a diverse patients group; SERS spectra are obtained from these specimens so that an appropriate population of both positive and negative SERS spectra are acquired, which can be thousands, tens of thousands, or millions of SERS spectra; these SERS spectra and associated infectious status of the specimen will be used as an input and validation databases to train and optimize the deep learning model; and once validated, the the deep learning model and the measured SERS spectra (1, 2, or even 20 s, or 30 s) are used in conjunction with the new spectra from the patient as input to establish a threshold and determine the infectious status of the patient's specimen.

SERS systems and methods of the present disclosure can be used to detect a purified biological agent, as well as a biological agent present in different types of samples. The samples can include blood, saliva, tears, phlegm, sweat, urine, plasma, lymph, spinal fluid, cells, microorganisms, aqueous dilutions thereof, or a combination thereof that can be acquired from a subject (e.g., mammal such as a human). In further aspects, the sample can be obtained from human nasopharyngeal swabs. The sample can contain no biological agents of interest, a single biological agent of interest, or multiple biological agents of interest. In some aspects, a biological agent “of interest” is a biological agent being detected in a sample. The biological agent can include different types of viruses. In further aspects, the virus can be a member of the subfamily Orthocoronavirinae. In more particular aspects, the virus can be SARS-CoV-2 or a variant thereof.

Now having described the present disclosure, additional details are provided in Example 1.

### Example 1

### Introduction

Recently, surface-enhanced Raman spectroscopy (SERS) has been extensively explored as a potential diagnostic platform for detecting SARS-CoV-2, owing to its remarkable sensitivity, ability to provide unique “signature” spectral features for different viruses, inherent simplicity, and capability for a point-of-care detection device. [1, 2] The overarching goal of SERS-based SARS-CoV-2 detection is to establish a portable alternative to the current gold standard, the reverse-transcription real-time polymerase chain reaction (RT-PCR) technique. Various detection strategies have emerged, such as direct detection of viral particles, [3-5] RNA [6, 7] and spike proteins capture and detection, [8] as well as SERS label-based approaches [9, 10]. Notably, direct detection methods have gained prominence, primarily owing to the inherent advantages of SERS technique, and their performance reported in the literature is summarized as Table 1. These methods can be categorized into three distinct aspects: classification, quantification, and a combination of classification and quantification.

TABLE 1
Summary of SERS (or Raman)-based direct detection of SARS-CoV-2.

| | Classification | Quantification | Detection | | | |
|---|---|---|---|---|---|---|
| Virus | method | method | range | LOD | Media | Ref. |
| SARS-CoV-2, | PCA, RF | NA | NA | NA | Saliva | 1 |
| H1N1 A, | | | | | | |
| Marburg, Zika | | | | | | |
| virus | | | | | | |
| SARS-CoV-2 | SVM | NA | NA | NA | | 2 |
| SARS-CoV-2 | LDA | NA | NA | NA | Saliva, serum | 3 |
| human | | | | | | |
| adenovirus | | | | | | |
| type 7, and | | | | | | |
| H1N1 virus | | | | | | |
| SARS-CoV-2, | Gradient | NA | NA | NA | Spiked clinical | 4 |
| human | boosting | | | | nasal swab | |
| coronaviruses | machine | | | | | |
| OC43, NL63, | | | | | | |
| 229E, | | | | | | |
| Influenza A | | | | | | |
| (H1N1), | | | | | | |
| respiratory | | | | | | |
| syncytial virus, | | | | | | |
| and | | | | | | |
| Streptococcus | | | | | | |
| pyogenes | | | | | | |
| SARS-CoV-2- | Multivariate | NA | NA | NA | Serum | 5 |
| induced serum | and | | | | (clinic) | |
| metabolic | univariate | | | | | |
| profiles | analyses | | | | | |
| SARS-CoV-2 | PCA, SVM | NA | NA | NA | Saliva | 6 |
| | (95%) | | | | (clinic) | |
| SARS-CoV-2 | RNN | NA | NA | NA | Throat swab or | 7 |
| | (87.7%) | | | | sputum | |
| | | | | | (clinic) | |
| SARS-CoV-2 | 1D-CNN | NA | NA | NA | Saliva | 8 |
| | (89-92%) | | | | (clinic) | |
| SARS-CoV-2, | PCA | Calibration | 2 × 10³-1 × 10⁴ | 100 | Saliva, | 9 |
| human | | curve | copies/test | copies/test | serum | |
| adenovirus 3 | | | | (PFU/test) | | |
| and H1N1 | | | | | | |
| influenza | | | | | | |
| virus | | | | | | |
| SARS-Cov-2 | NA | Calibration | 10⁻²-10³ | 10⁻² | Nasopharyngeal | 10 |
| | | curve (with | PFU/mL | PFU/mL | swab | |
| | | Ct value) | | | (clinic) | |
| SARS-CoV-2 | NA | Calibration | 10⁻¹-10³ | 10⁻² | Nasopharyngeal | 11 |
| | | curve (with | PFU/mL | PFU/mL | swab | |
| | | Ct value) | | | (clinic) | |
| Thirteen | SVM | SVR | 190-10⁵ | ~190 | Spiked Saliva | 12 |
| respiratory | | | PFU/mL | PFU/mL | | |
| viruses | | | | | | |
| SARS-CoV-2 | NA | Autoencoder | 10¹-10⁴ | 10¹ | Artificial breath | 13 |
| | | | PFU/mL | PFU/mL | aerosols | |
| SARS-CoV-2 | RNN | RNN based | 10.61-36.51 | 10.61 (Ct | nasopharyngeal | This |
| | (98.5%) | regression | (Ct values) | value) | swab | work |
| | Blind test | (Ct value) | | | (clinic) | |
| | (99.02%) | | | | | |

Abbreviations:
PCA: principal component analysis,
RF: random forest,
SVM: support vector machine,
LDA: linear discriminant analysis,
CNN: convolutional neural network,
RNN: recurrent neural networks

Classification, a pivotal aspect of COVID-19 management and intervention, involves determining the SARS-CoV-2 status of specimens. As specimens are derived from either spiked buffer solutions or body fluids, inherent background SERS signals are presented in all measured spectra, leading to significant interference. To counter this challenge, advanced spectral analysis techniques, particularly machine-learning and deep-learning algorithms (MLAs and DLAs), have been widely employed for the classification of SERS spectra. [4, 11-14]A noteworthy example is the successful differentiation of non-infectious lysed SARS-CoV-2 using support vector machine (SVM) analysis based on SERS spectra. [15] The distinction between SARS-CoV-2, A/influenza (H1N1), Marburg, and Zika viruses in spiked saliva has been achieved through a random forest (RF) algorithm, yielding varying accuracies from 85.4% to 95.6% [4]. Recent advancements showcase the potential for SERS spectra-based SVM classification, attaining >99% accuracy in detecting thirteen distinct respiratory viruses in saliva, including two SARS-CoV-2 variants. [3] So far, only a limited number of studies have centered on classification of real clinical specimen [11-14]. For instance, SERS spectra collected from patients' saliva samples were subjected to a SVM classifier, achieving a prediction accuracy of 95% for differentiating positive and negative COVID-19 cases. [13] Similarly, a residual neural network-based approach was used for the detection of the SARS-CoV-2 S antigen from clinical throat swab or sputum specimens, demonstrating an 87.7% accuracy. [14] Notably, the majority of these studies showcased classification performance by gathering SERS spectra from fewer than 30 patients, yielding accuracy levels spanning from 87.7% to 95%.

The literature showcases two distinct methodologies for quantifying SARS-CoV-2: the calibration curve method [16-18] and the regression method based on MLA or DLA [3, 19]. In the calibration curve method, the SERS intensity of a specific peak, uniquely linked to a particular virus, is plotted against its concentration. For instance, Ansah et al. presented a calibration curve for the SARS-CoV-2 detection in saliva on the intensity of SERS peak at 732 cm⁻¹ or 964 cm⁻¹ [18]. However, when quantifying clinical specimens, at least two challenges must be addressed. Firstly, based on our recent study on 13 respiratory viruses [3], most spectral features of SARS-CoV-2 are shared with other viruses (refer to Table S11 of Ref. [3]). This phenomenon is expected, given that the spike protein of SARS-CoV-2, contributing dominantly in SERS spectra, shares a comparable composition and structure with spike proteins from other viruses. [20-22] Secondly, the interference in the SERS spectra from background, stemming from buffers or body fluids, is significant. These backgrounds tend to overshadow spectral features from viruses, compared the relative contents of virus and specimen medium. Our previous SERS investigation on virus-spiked saliva with varied viral concentrations did not yield a straightforward correlation between SERS peak intensity and virus concentration[3]. Yet, a meticulous examination of spectra across diverse viral concentrations unveiled subtle spectral alterations. To surmount these challenges and glean quantification insights, we employed SVM-based regression to establish quantitative calibration curves for eleven respiratory viruses, accurately estimating unknown virus concentrations in buffer and saliva within a detection range of approximately 195−1×10⁵ PFU/mL [3]. In a parallel endeavor, Hwang et al. developed a DLA-based autoencoder, followed by the targeted elimination of non-discriminatory SERS features of spike proteins, facilitating the quantification of 10¹−10⁴ PFU/mL SARS-CoV-2 lysates in aerosols with an accuracy surpassing 98% [19].

Undoubtedly, to bring SERS-based diagnostic methodologies in line with the rigor of RT-PCR techniques, it becomes essential to establish three crucial attributes, while capitalizing on the inherent benefits of SERS: Firstly, achieving effective classification of specimens as either SARS-CoV-2 positive or negative; secondly, attaining accurate quantification of the SARS-CoV-2 viral load, specifically the cycle threshold (Ct) value; and finally, successfully detecting the virus within genuine clinical specimens. Up until now, there exists no report that combines SERS with MLA or DLA to classify and quantify SARS-CoV-2 infection from real clinical specimens and subsequently performs a direct comparison with RT-PCR results.

This study directly compares the use of SERS and DLA for detecting and quantifying SARS-CoV-2 in clinical HNS specimens to the results from RT-PCR. The process involves three steps: mixing an HNS specimen with a virus inactivation buffer, placing a droplet on a SiO₂-coated silver nanorod array (AgNR@SiO₂) SERS substrate, and collecting multiple SERS spectra from different substrate locations after drying. Two DLAs, both based on recurrent neural networks (RNNs), are constructed: one for classification and the other for regression. The classification model distinguishes positive and negative HNS specimens with a remarkable 98.5% accuracy, while the regression model predicts RT-PCR Ct values with an average root mean square error (RMSE) of 1.627. Notably, both tasks depend on inherent SERS spectral differences in viral components. Blind tests on 104 unknown HNS specimens show that the SERS-DLA approach achieves 98.28% accuracy for positive specimens and 100% accuracy for negative ones. Ct values are predicted with a small RMSE of around 1.3. These outcomes demonstrate the comparable performance of SERS-DLA to RT-PCR, providing a direct, rapid, and reliable point-of-care COVID-19 diagnostics platform.

### Experimental Section

Materials. Silver (Kurt J. Lesker, 99.999%) and titanium pellets (Kurt J. Lesker, 99.995%) were purchased as evaporation materials. Tetraethylorthosilicate (TEOS; Alfa Aesar, 99.9%), ammonium hydroxide (J. T. Baker, 28.0-30.0 wt. %) and ethanol (EtOH; Sigma-Aldrich, 95%) were used for silica coating on AgNR. Guanidine hydrochloride, Triton X-100, EDTA, and Tris-HCl were obtained from Sigma and used for preparing virus inactivation buffer. Polydimethylsiloxane (Sylgard 184, PDMS) was purchased from Dow Corning. Pure water (Sigma-Aldrich) was used throughout all the experiments. All the reagents were used without further purification.

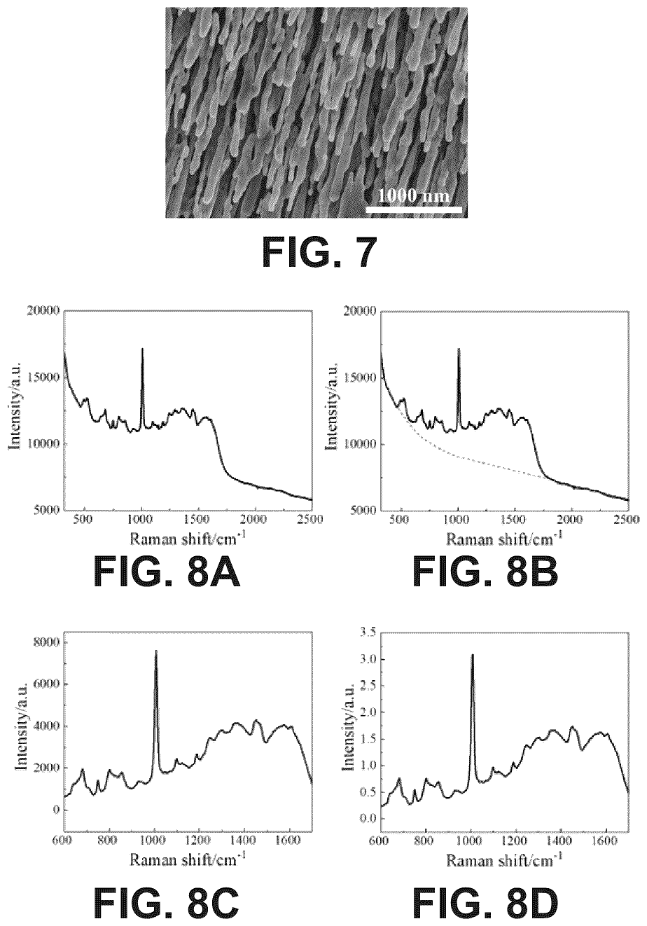

AgNR@SiO₂ arrays fabrication. AgNR@SiO₂ SERS substrates were prepared by the oblique angle deposition (OAD) and salinization via hydrolysis of TEOS as described previously [23, 24]. The AgNR substrates were first prepared using OAD according to Ref. [25, 26]. Piranha solution cleaned glass slides (0.5 inch×0.5 inch) were mounted in a custom-designed electron beam deposition system. A layer of 20 nm Ti film and a layer of 100 nm Ag film were subsequently deposited at a rate of 0.2 nm/s and 0.3 nm/s, respectively. Then, the vapor incident angle was adjusted to be 86°, and a thickness of 2000 nm Ag film was deposited at a rate of 0.3 nm/s to form the AgNRs on the substrates. The entire evaporation process was conducted under a high vacuum condition (chamber pressure <3×10⁻⁶ Torr). After the deposition, the AgNR substrates were immersed into a homogeneous mixture of 30 mL of EtOH, 4 mL of H₂O, and 500 μL of TEOS for 20 min under stirring. The coating of SiO₂ was initiated after adding 560 μL of ammonium hydroxide. The substrates were removed from the reaction solution after 5 min, followed by water rinsing and N₂ drying. A 2-nm conformal SiO₂ coating on AgNR was expected under such conditions. Subsequently, arrayed small wells (4 wells, with a well diameter of 4 mm and a well depth of 1 mm) on a PDMS layer were molded on the AgNR@SiO₂ array to restrict the effective sensing areas [27], and we refer them as AgNR@SiO₂ wells. A typical scanning electron microscopy (SEM) image of the AgNR@SiO₂ array is shown in FIG. 7.

Patient HNS specimens. Deidentified HNS specimens were obtained from the University of Georgia Veterinary Diagnostic Laboratories (GVDL) for this study. These specimens were residual samples from the Clinical Laboratory Improvement Amendments (CLIA)-registered GVDL's confirmatory RT-PCR diagnostic testing. HNS specimens were collected using a sterile swab applicator and placed in 1 mL of saline. The GVDL determined the SARS-CoV-2 status of each HNS specimen using an Applied Biosystems TaqPath COVID-19 Combo kit EUA assay (ThermoFisher catalog number A47814) in a multiplex RT-PCR format. The multiplex RT-PCR assay had 3 target gene fragments: spike (S), nucleocapsid (N), and Orf1 ab (ORF1 ab) protein regions, which exhibit high specificity and low risk for mutation (except for the S gene). The RT-PCR data are analyzed and then interpreted by the Applied Biosystems™ COVID 19 Interpretive Software. For the positive specimens, the corresponding Ct values for three viral gene fragments were recorded. 120 SARS-CoV-2 positive and 120 negative specimens were used for SERS spectra collection following the procedure: A 30 μL aliquot of the collected HNS specimen was mixed 1:1 (v:v) with the inactivation buffer containing 1 M guanidine hydrochloride, 0.2% Triton X-100, 1 mM EDTA, and 2 M Tris-HCl with a pH=7.8, followed by room temperature incubation for 5 min. Then the mixture (10 μL) was diluted by 300 μL pure water without further processing. It is expected that under this treatment, the SARS-CoV-2 viruses in positive HNS specimens were inactivated. All the experiments were carried out in a BSL-2 lab.

SERS measurements. For SERS measurements, 20 μL of above specimen lysate was dispensed onto a AgNR@SiO₂ well, and was incubated for 5 min. Then the well was washed with DI water (X3) and air-dried at 20° C. (the drying time varied from 2 min to 5 min). The SERS spectra were acquired by using a Tec5USA Raman spectroscopy (Tec5USA Inc.), with a 785 nm excitation laser with a beam diameter of ˜100 μm, a power of 35 mW, and an acquisition time of 4 s. 20 SERS spectra were collected from randomly selected locations in each well.

Machine learning and deep learning algorithms for classification. Five different algorithms, including SVM, RF, back-propagation (BP), convolutional neural network (CNN), and RNN, were applied for classifying the patient HNS specimens based on the SERS spectra. The total spectral set included 2400 SERS spectra collected from 120 positive and 2400 SERS spectra from 120 negative specimens. All the original SERS spectra were preprocessed following a procedure described below [28], which includes a baseline removal and a spectrum normalization. The entire spectral set was shuffled randomly to make the spectral set have both positive and negative specimen data in each batch. The entire spectral set was split into 70%: 15%: 15% for training set, validation set, and test set, respectively. The RNN model had one convolutional layer (the size of the convolutional kernel was 5×1 and the number of filters was 32), one pool layer (Max Pool), three consecutive blocks, i.e., one convolutional block (Conv block) and two identity (ID) blocks (the convolutional kernel size of the two blocks were 5×1 and 7×1, respectively, and the number of filters was 32 and 64, respectively), two long-short term memory (LSTM) layers with 1400 and 300 units, and one fully connected layer. The learning rate of the Adam optimization algorithm was 0.03. The other four classification algorithms, SVM, RF, BP, CNN, are detailed below. SVM and RF analyses were performed in MATLAB and GridSearch was used to optimize the parameters. The BP, CNN, and RNN models were performed using Tensorflow 2.4 environment of PyCharm Community software. In these models, the batches and number of iterations of the training instances (spectra) were set to be 20 and 207, respectively.

Machine learning and deep learning algorithms for regression. The RNN regression model included two LSTM layers, two dropout layers, and three fully connected layers. The sizes of the LSTM layers were 700 and 300, with dropout sizes of 0.5 and 0.3, and the sizes of the fully connected layers were 500, 200, and 100, with L1 loss used as the loss function, Adam as the optimizer, and a learning rate of 0.001. The other regression algorithms based on SVM, RF, BP, and CNN are detailed below.

Blind tests. 104 extra deidentified HNS specimens with 46 positives and 58 negatives were determined by RT-PCR test. These specimens were given to the operator for the SERS blind test, without informing the SARS-CoV-2 status of each specimen. A total of 21 SERS spectra were measured from each specimen at different locations. These newly obtained SERS spectra were used as input in the previously trained RNN models to predict the infection status of each specimen. A ratio γ

$$( γ = \frac{{n}_{-}}{{n}_{+} + {n}_{-}} )$$

is defined to predict the SARS-CoV-2 infection status and the subsequence Ct value of the positive specimen is predicted by the RNN regression model. Here n₊ (n₋) is the number of positive (negative) predictions among the 21 spectra obtained from one specimen. A threshold of γ=0.7 is used based on the RNN model result, which means that the specimen is SARS-CoV-2 negative when γ≥0.7 and positive when γ<0.7. The accuracy of the classification RNN model was verified by comparing the predicted infection status by the RNN model with the status of the corresponding specimen previously determined by RT-PCR. For the Ct value prediction of positive specimens, the average spectrum of 21 spectra for each positive specimen was calculated and used as the input for the RNN regression model to obtain the predicted Ct values. The quantification accuracy of the blind test was verified by comparing the predicted Ct values with the corresponding Ct values determined by RT-PCR.

### Results and Discussion

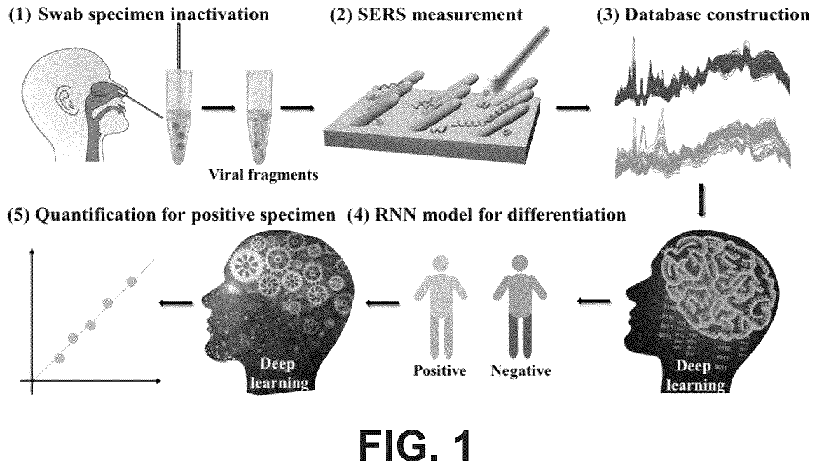

General detection and classification strategy. The procedure to use SERS and DLAs to directly differentiate and quantify SARS-CoV-2 positive and negative HNS specimens is illustrated in FIG. 1. The detection strategy includes of background and forward efforts. The background effort includes establishing SERS spectral database from known SARS-CoV-2 positive and negative HNS specimens and developing DLAs according to the spectral database. The forward effort is to use the established DLAs to validate the SARS-CoV-2 detection, i.e., a blind test. The background effort includes four steps: first, HNS specimens are collected in a viral inactivation buffer so all the operations can be performed in BSL-2 lab. Second, the inactivated HNS specimens are dispended on AgNR@SiO₂ substrates for intense SERS measurements. After repeated SERS spectra are collected from different locations of the substrates and from known positive and negative specimens, an appropriate baseline correction method and spectral normalization are applied to all spectra. Finally, two RNN-based DLAs for classification and quantification are developed, the corresponding model parameters are optimized, and the models are cross-validated and tested. These DLA models with optimized parameters are used for blind tests.

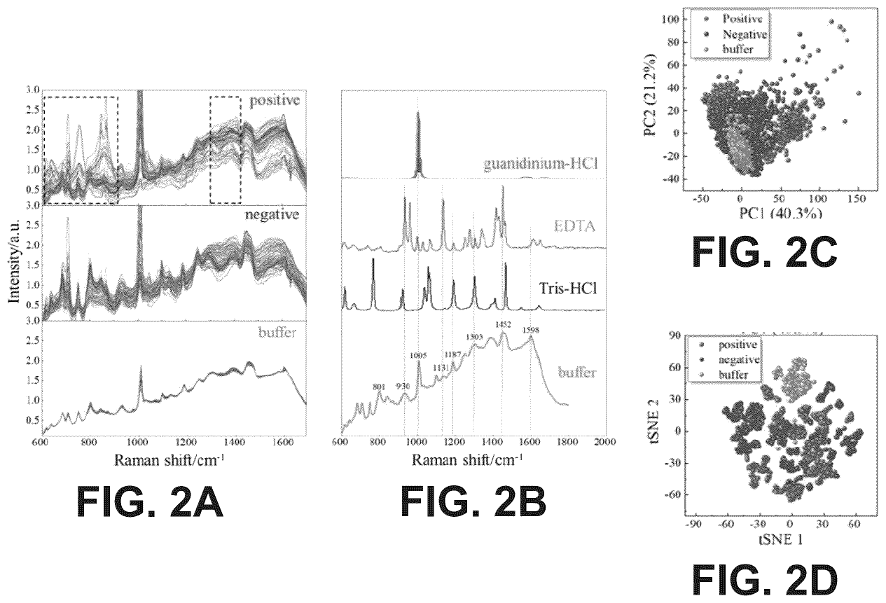

The SERS Spectra of HNS specimens. FIG. 2A (with complete spectra in FIG. 9) plots the baseline-corrected and normalized SERS spectra for 120 positive and 120 negative HNS specimens, as well as the inactivation buffer. The SERS spectra of the inactivation buffer exhibit clear and consistent peak features at specific wavenumbers (Δv=930, 1005, 1187, 1303, 1452, and 1598 cm⁻¹), attributed to various molecular compositions like guanidinium-HCl, Tris-HCl, and EDTA within the buffer. Such a consistency is reflected in FIG. 2B, where the primary peaks of the inactivation buffer align well with these composition-related SERS peaks. While the SERS spectra of both positive and negative HNS specimens share similar overall features with the inactivation buffer, especially when considering the average spectra (shown in FIG. 10), they exhibit more fluctuations. The inactivation buffer is commonly used in viral RNA purification by dissociating the virus into nucleic acid and protein fragments [29, 30]. Within the buffer, the chaotropic agent guanidinium-HCl can unfold proteins and break into original polypeptide chains. The viral proteins can be dissolved by detergent (Triton X-100) micelles as the viral lipid envelope is destroyed [31]. Therefore, it is expected that after inactivation, the SARS-CoV-2 viruses in the HNS specimens are decomposed into various compositions, such as proteins, RNAs, membrane, etc., as illustrated in FIG. 1 Step 1. Thus, the obtained SERS spectra from the positive and negative HNS specimens may have three contributions: (1) inactivation buffer, (2) HNS compositions, and (3) viral fragments in positive specimens. The molecules in inactivation buffer, being small, can easily adsorb to SERS “hot spots”, while viral fragments and HNS compositions, larger in size, face challenges in adsorption by these “hot spots”. Additionally, the buffer quantity largely outweighs other constituents during sampling.

Therefore, the SERS spectra of inactivated specimens shall be dominated by the spectral features from the buffer, resulting in high similarity spectral shapes from positive and negative specimens as well as the buffer. However, a comparison between the SERS spectra from positive and negative HNS specimens indicates that spectra from negative specimens exhibit greater uniformity and fewer variations. Conversely, spectra of positive specimens show considerable fluctuations in the 600-900 cm⁻¹ and 1300-1425 cm⁻¹ ranges. Given the striking similarity among these three sets of spectra, as illustrated in both FIGS. 2A and S4, traditional chemometric techniques like principal component analysis (PCA) and t-distributed stochastic neighbor embedding (tSNE) are difficult to discern the minute differences. FIGS. 2C and 2D present the PCA and tSNE plots, and all three clusters due to 3 sets of SERS spectra are intertwined together. Hence, simple classification methods are inadequate to differentiate the SERS spectra from inactivated HNS specimens. This prompts the introduction of MLAs and DLAs.

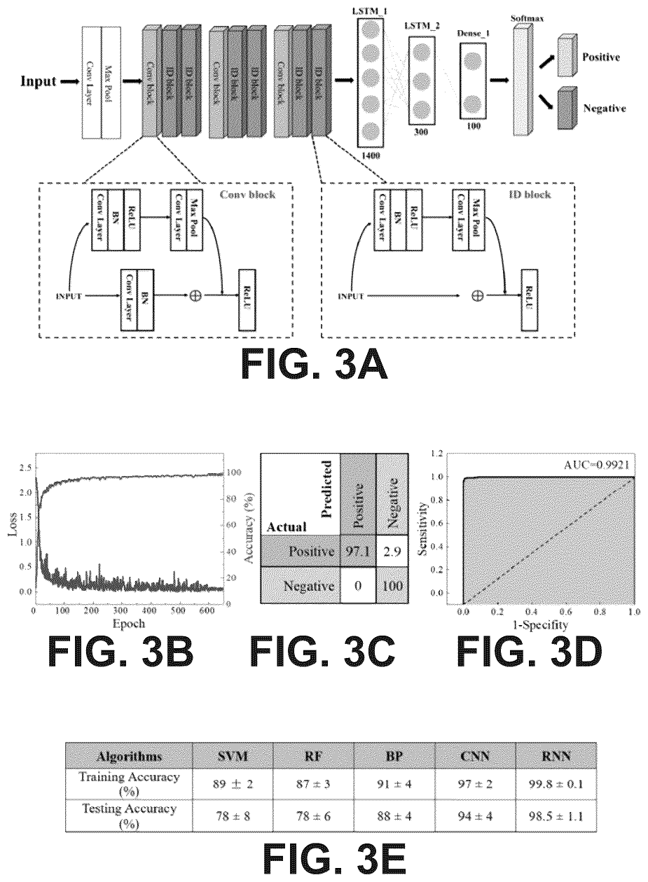

Deep learning model to classify SERS spectra of positive and negative specimens. An RNN-based deep learning model was developed to predict the SARS-CoV-2 status (positive or negative) of HNS specimens based on their SERS spectra. RNN architecture allows for cyclic connections between nodes and enable outputs from certain nodes to influence subsequent inputs to those same nodes. [32] This property is particularly useful for handling variable-length sequence inputs, such as SERS spectra. FIG. 3A shows the architecture of our RNN model. A convolutional layer (Conv Layer) and a maximum pool (Max Pool) were first used to preprocess the input spectra. Then three consecutive blocks composed of a convolutional block (Conv block) and two identity (ID) blocks with shortcuts were connected to the previous Max Pool. The Conv block included an initial Conv layer, followed by a batch normalization (BN), a corrected linear (ReLU) transform, and a Max Pool. The ID block had a similar framework without the Conv Layer and BN after the input. The Conv block and ID block can solve the problem of gradient disappearance of deep structures by marking shortcut connections from previous input data to output data to maintain previous gradient information, reduce the computational power, and increase the training efficiency. Two LSTM layers and a dense layer were added after three consecutive blocks. These elements can effectively store long-term states and overcome challenges associated with gradient vanishing and gradient explosion during extended sequence training. The output layer utilized a Softmax function as its activation function, contributing to the final predictions.

The 4800 SERS spectra of positive and negative HNS specimens were trimmed, excluding the spectral features from 960 to 1080 cm⁻¹. This exclusion was driven by two factors: substantial fluctuations in normalized peak intensity (illustrated in FIG. 9); and the dominant presence of the peak at Δv=1005 cm⁻¹ due to inactivation buffer. So only 982 one-dimensional float spectral data from 600-960 cm⁻¹ and 1080-1700 cm⁻¹ per spectrum, were retained. In the binary classification, positive and negative HNS specimens were denoted as 0 and 1, respectively. In any neural networks, the overfitting issue is one of the major concerns due to excessive correspondence to specific dataset. [32] To mitigate overfitting, 70% of the entire spectral set was employed for training, 15% for validation, and another 15% for testing. Different RNN units, including simple RNN, gated recurrent unit (GRU), and bidirectional RNN, were initially compared to select the optimal unit.

Subsequently, the chosen RNN model underwent optimization involving hyperparameter tuning (e.g., various optimizers, learning rates, loss functions, and fully connected layers), as detailed in Table 2. With optimized hyperparameters, the spectral set was trained with increasing epochs.

TABLE 2
Recurrent neural network (RNN) model parameter settings.

| Input | LSTM1 | LSTM2 | | Batch | Learning | |
|---|---|---|---|---|---|---|
| size | size | size | Bias | first | rate | Bidirectional |
| 1400 | 1400 | 300 | True | False | 0.03 | True |

TABLE 3
CNN parameter settings.

| Input | | | | | | Loss | |
|---|---|---|---|---|---|---|---|
| size | Kernel | Filters | Stride | Padding | Pooling | function | Learning rate |
| 1400 | 3 × 1, 3 × 1, | 8, 8, 32, | 1 | 2 | Max | Cross- | 0.002 |
| | 5 × 1, 5 × 1, 7 × | 32, 64, | | | Pooling | entropy | |
| | 1, and 7 × 1 | and 64 | | | | | |

FIG. 3B shows the evolution of the loss function and accuracy against epochs for the test spectral set. The loss function rapidly decreased from 2.3 to 0.2 until around 100 epochs, subsequently stabilizing at approximately 0.025 after 200 epochs. Meanwhile, the accuracy of the test spectral set rose from 12% to 80%, eventually converging to 95%. At around 650 epochs, the accuracy reached 98.5%, marking the conclusion of training. The RNN model's performance was evaluated using a confusion matrix (FIG. 3C) based on the test spectral set. It achieved a 97.1% accuracy for positive spectra and a perfect 100% accuracy for negative spectra. The false-negative prediction for positive specimens could be due to non-uniform distribution of viral component on the SERS substrate from the trace amount of virus in the specimens. The receiver operating characteristic (ROC) curve (FIG. 3D) yielded an area under curve (AUC) of 0.9921, indicating outstanding classification performance close to unity. In comparison to other MLAs (SVM and RF) and DLAs (BP and CNN, details below), the RNN model excelled. As summarized in FIG. 3E, the RNN model exhibited the highest training and testing accuracies, reaching 99.8% and 98.5%, respectively. This demonstrated its superior classification performance relative to other models like SVM, RF, CNN, and BP.

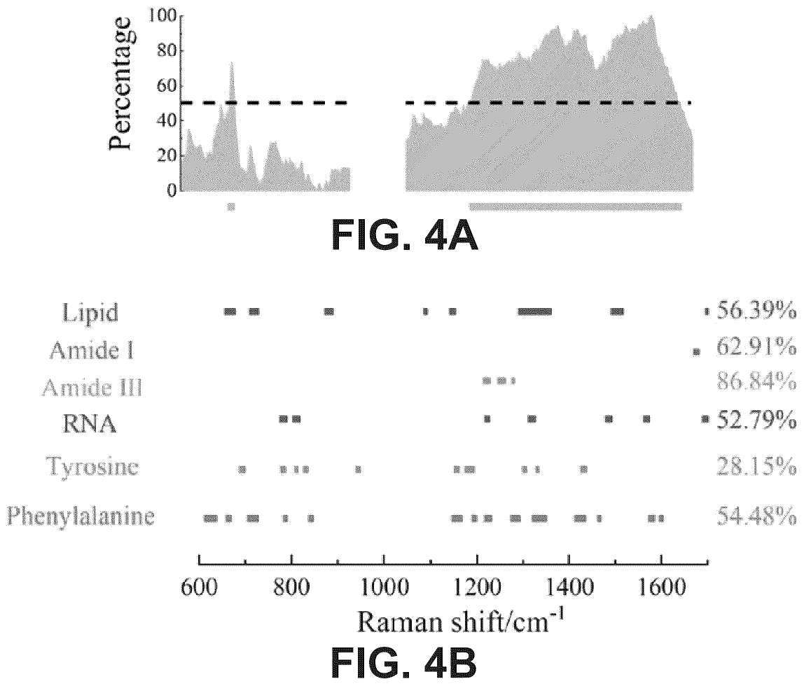

Given the striking similarity between positive and negative spectra in FIG. 2A, comprehending the source of such high classification accuracy in the RNN model becomes crucial. A method to achieve this understanding is through a “feature importance map (FIM),” which assigns higher importance values to specific wavenumbers based on their contributions in the RNN model's identification of SARS-CoV-2. Here the calculation of the FIM is based on a full-gradients algorithm [5, 33], elaborated below. FIG. 4A shows the resulting FIM based on the RNN model, and spectral features spanning 699-717 cm⁻¹ and 1219-1678 cm⁻¹ ranges emerge as key signatures in the RNN model's classification process. By cross-referencing the FIM to existing Raman spectra of known biomolecules and chemical functional groups such as lipids, proteins, nucleic acids, amino acids, and amides, we can have a better understand which composition contributes the most in the RNN model to differentiate SARS-CoV-2 positive specimens. A “matching score” is proposed for this purpose[5]: First, an important wavenumber range contributing to the SERS spectra's classification (above the 50% threshold) was extracted from the FIM, denoted as R_{RNN}. The purple lines beneath the FIM in FIG. 4A indicate the significant R_{RNN} range surpassing the 50% threshold. Then, the wavenumber ranges of the Raman peaks of lipid, amide I, amide III, RNA, tyrosine, and phenylalanine were obtained from previous reports [34, 35], and the corresponding wavenumber ranges of the Raman peaks are designated as R_{K}, which are presented as different colored segmented lines in FIG. 4B. The extent of overlapping wavenumbers for each biomolecule between R_{RNN} and R_{K} was calculated as R_{RNN} ∩R_{K}, where “∩” denotes overlap. The matching score was then defined by the ratio of R_{RNN-K} and R_{K}, i.e.,

$$matching score = \frac{{R}_{R N N} ∩ {R}_{K}}{{R}_{K}} .$$

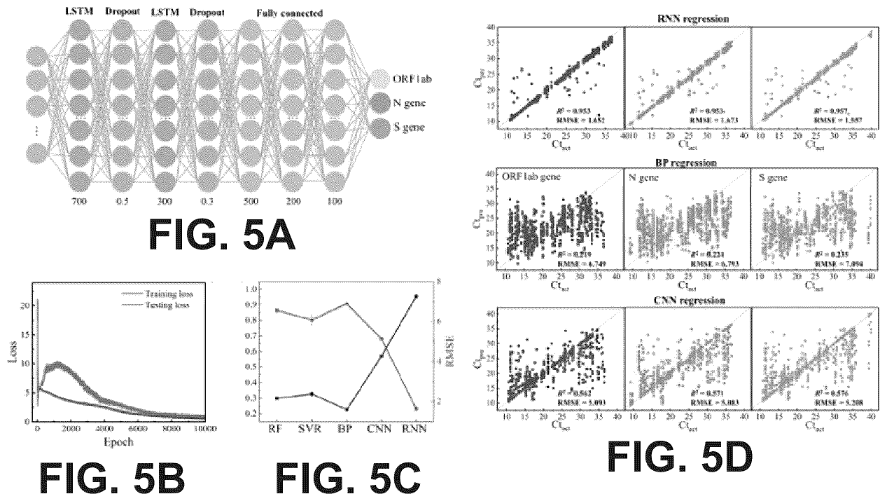

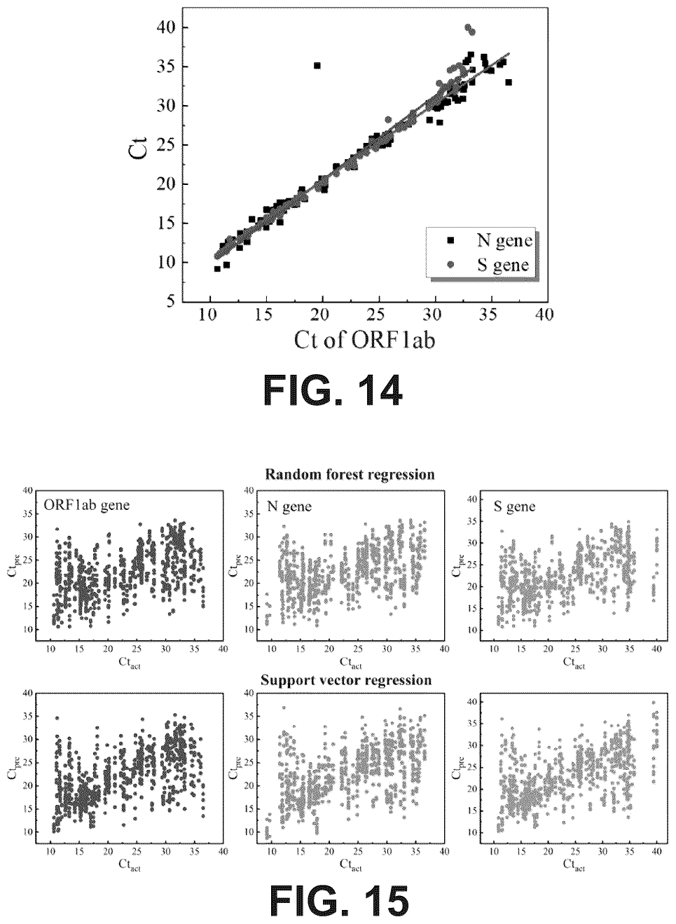

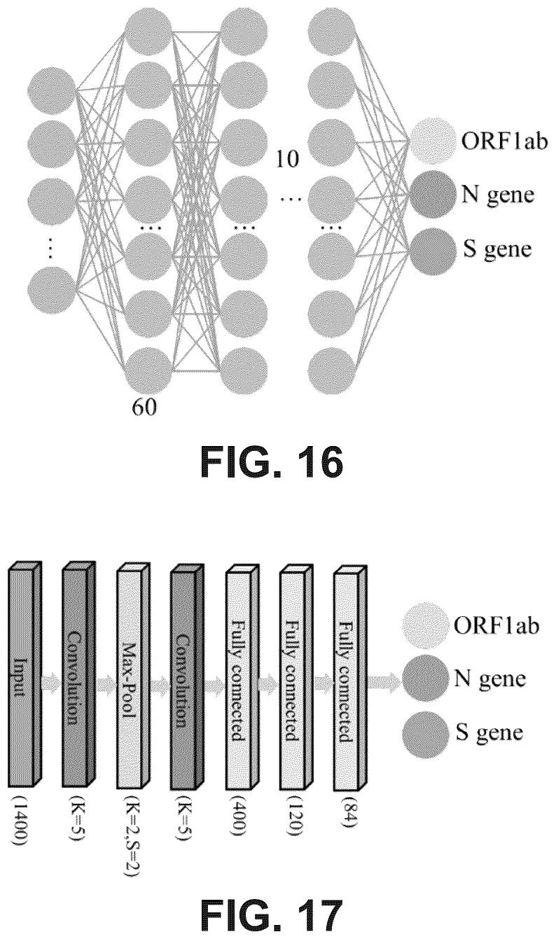

A higher matching score indicates a greater likelihood of the specific biomolecule's Raman signatures contributing to distinguishing negative and positive SERS spectra during testing. The calculated matching score of each biomolecule is indicated as a percentage in FIG. 4B. Notably, amide III and amide I exhibit high matching scores of 86.84% and 62.91%, signifying their crucial role in distinguishing SARS-CoV-2 spectra, followed by lipid (56.39%), phenylalanine (54.48%), and RNA (52.79%). As SARS-CoV-2 is enveloped in a phospholipid bilayer, lipid also holds a substantial matching score of 56.39%. The RNN model appears to leverage the signature ranges of phenylalanine and RNA for the differentiation of positive and negative spectra. SARS-CoV-2 virion has roughly spherical or ellipsoidal shape with an average diameter of 108±8 nm. It has a positive-sense and single-stranded RNA genome about 30,000 bases long, which encodes the virus structures. Within the lumen of the virion are the ribonucleoprotein (RNP) complexes including the nucleocapsid protein (N) and the viral genome, responsible for the packaging of the RNA genome of the virus. Virus envelope, made of lipid bilayer, is anchored with membrane proteins (M), envelope proteins (E) and spike structural proteins in the ratio around 1:20:300. [36, 37] The quantification of viral load. A prevalent output from RT-PCR is the Ct value, which signifies the cycles needed for the fluorescent signal to surpass a predefined threshold, confirming the target nucleic acid's presence. This Ct value inversely correlates with the nucleic acid's quantity—a lower Ct indicates a higher nucleic acid amount [38] and can be used as a semi-quantitative measure of the amount of viral RNA in the specimen, as discussed below. Therefore, to compare SERS-DLA based detection with RT-PCR, it is important to predict the corresponding Ct value in positive specimens. Regression models were constructed for predicting Ct values of three viral gene fragments: ORF1 ab, N gene, and S gene, all obtained from positive specimens. FIG. 14 shows near-identical Ct values for these three genes. In some instances, mutations in the spike protein made the S gene's Ct value unavailable, and the missing Ct value was substituted with the average of ORF1 ab and N gene Ct values. The architecture of the proposed RNN regression model is shown in FIG. 5A, including two LSTM layers, two dropout layers, and three fully connected layers. The sizes of the LSTM layers are 700 and 300, with dropout sizes of 0.5 and 0.3; and the sizes of the fully connected layers are 500, 200, and 100, with L1 loss used as the loss function, Adam as the optimizer, and a learning rate of 0.001. Refer to Table 4 for details. The spectral set was divided into 70% for training and 30% for testing. Employing optimized hyperparameters, the loss functions of the training and test spectral set were recorded in FIG. 5B. The training set's loss function decreased sharply from 17.3 to 3.6 over the first 2000 epochs, while the test set's loss function exhibited fluctuations. At around 10000 epochs, the loss functions of the training and test sets stabilized around 0.6 and 1.1, respectively, indicating a gradual convergence of training.

Regression results for three viral gene fragments (ORF1 ab, N gene, and S gene) of the predicted Ct value and actual Ct value from the RNN regression model are plotted in FIG. 5D. The predicted and actual Ct values of all three viral gene fragments follow the relationship Ctₚᵣₑ=Ct_{act}, with an average small RMSE of 1.627 and the coefficient of determination R² of 0.955. These performance parameters underline the accuracy of the RNN regression model. Consider the specimen dilution, sample volume, and laser beam size, comparing to PCR, the LOD can lower four orders of magnitude as detailed below. The differentiation is also due to other virus components, e.g., lipid, viral protein, biomarkers related to SARS-CoV-2 infection in HNS specimen, e.g., inflammatory mediators, as well as breath volatile organic compounds (BVOCs) [39, 40].

TABLE 4
RNN regression model parameter settings.

| | | | Fully | | | | | |
|---|---|---|---|---|---|---|---|---|
| Input | LSTM1 | LSTM2 | connected | | | Batch | Learning | |
| size | size | size | layer | Dropout | Bias | first | rate | Bidirectional |
| 1400 | 700 | 300 | 500, 200, | 0.5, 0.3 | True | False | 0.002 | True |
| | | | 100 | | | | | |

TABLE 5
CNN regression model parameter settings.

| | | | | Fully | | |
|---|---|---|---|---|---|---|
| Input | | | | connected | Loss | Learning |
| size | Kernel | Stride | Pooling | layer | function | rate |
| 1400 | 5 × 1, 5 × 1, | 2 | Max | 400, 120, 84 | L1 loss | 0.002 |
| | 2 × 1 | | Pooling | | | |

The predictive capabilities of Ct values for three viral gene fragments using five regression models—RNN, CNN, BP, SVR, and RF—are compared: the BP and CNN results are presented in FIG. 5D, while those for RF and SVR are shown in FIG. 15. The corresponding RMSEs and R² values are summarized in Table 6 and FIG. 5C. SVR's if is approximately 0.299 with an RMSE of 6.544, while RF's if is roughly 0.328 with an RMSE of 6.072. For the two DLAs, the BP model yields an R² of approximately 0.226 with an average RMSE of 6.879, while the CNN regression model demonstrates an R² of around 0.570, accompanied by an average RMSE of 5.128. Notably, the RNN regression model stands out with the highest quantification performance, as depicted in FIGS. 5C and 5D. Evidently, SVM and RF, both falling under the MLA category, exhibited weaker predictive performance compared to DLAs like BP, CNN, and RNN. This difference results from DLAs' capacity to effectively learn intrinsic data features and optimize loss functions. DLAs excel in fitting intricate non-linear relationships, possessing a higher-dimensional hypothesis space and enhanced representation abilities. Conversely, MLAs rely on predetermined features and exhibit reduced generalization capabilities. In the realm of DLAs, the order of performance is as follows: BP<CNN<RNN. The RNN model's superior capabilities lie in its ability to process diverse input sequences through internal memory, allowing the hidden layer to correlate current and past data points for enhanced feature identification, particularly changes in SERS spectra trends. CNN and BP models, focusing on local intensity at distinct wavenumbers, disregard correlations among spectral features in neighboring wavenumbers. CNN's improved performance over BP arises from its reduced parameter requirements, lower model complexity, and optimized connection weights. Ultimately, the RNN model achieves the highest predictive accuracy, making it the preferred choice for blind test of patient specimens.

TABLE 6
Performance of 5 different regression algorithms.

| Algorithms | Gene name | R² | MSE | RMSE | MAE |
|---|---|---|---|---|---|
| Random | ORF1ab | 0.2989 | 40.8681 | 6.3928 | 4.8038 |
| forest | N gene | 0.2962 | 41.8439 | 6.4687 | 4.9019 |
| regression | S gene | 0.3033 | 45.8510 | 6.7713 | 5.1031 |
| Support | ORF1ab | 0.3144 | 33.4610 | 6.57845 | 4.2199 |
| vector | N gene | 0.3443 | 34.9284 | 5.9145 | 4.4195 |
| regression | S gene | 0.3242 | 42.7656 | 5.7241 | 4.6440 |
| BP | ORF1ab | 0.2185 | 45.5539 | 6.7494 | 5.0717 |
| regression | N gene | 0.2239 | 46.1442 | 6.7930 | 5.1207 |
| | S gene | 0.2353 | 50.3250 | 7.0940 | 5.3091 |
| CNN | ORF1ab | 0.5618 | 25.9384 | 5.0930 | 2.9478 |
| regression | N gene | 0.5714 | 25.8394 | 5.0832 | 2.9995 |
| | S gene | 0.5761 | 27.1244 | 5.2081 | 3.0453 |
| RNN | ORF1ab | 0.9534 | 2.7293 | 1.6521 | 0.5655 |
| regression | N gene | 0.9530 | 2.7988 | 1.6733 | 0.5737 |
| | S gene | 0.9573 | 2.4243 | 1.5565 | 0.5425 |

MSE: mean squared error.
RMSE: root mean square error.
MAE: mean absolute error.

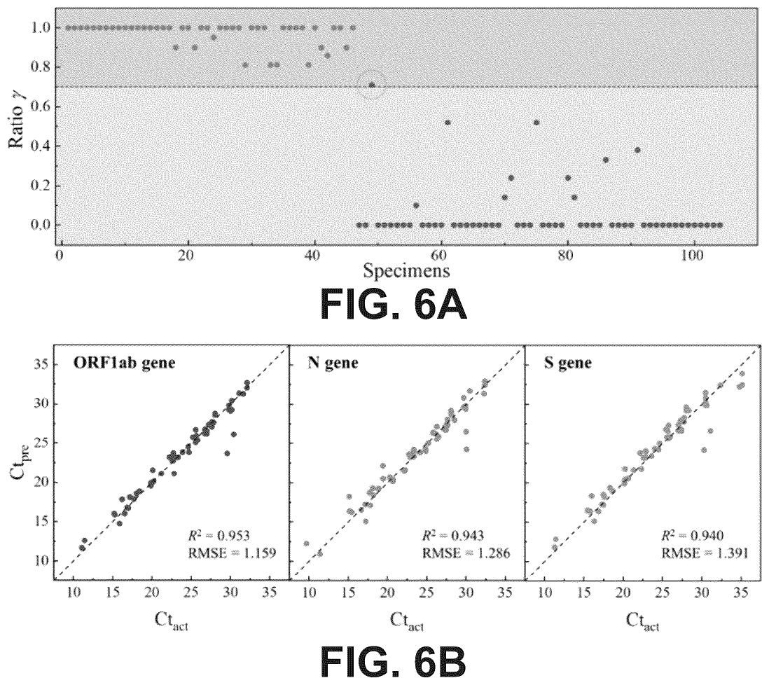

Blind SARS-CoV-2 diagnosis with RNN model. We assessed the applicability of the established RNN model for predicting the status of 104 deidentified HNS specimens in a blind test, comprising 46 negatives and 58 positives. The SARS-CoV-2 status of these specimens, as determined by RT-PCR tests, remained concealed from the SERS operator. The blind test procedure and assessment criteria were outlined in the experimental section. The SERS spectra from these specimens constituted the test spectral set, subjected to the previously trained RNN model to predict the positive or negative nature of each spectrum. A threshold value of 0.7 for γ was chosen, because as indicated in FIG. 3C, the RNN model exhibited a false-negative prediction rate of 2.9% and a false-positive prediction rate of 0%. The obtained classification results from the SERS spectra were then compared to the RT-PCR determined status of each corresponding blind specimen. Table 7 summarizes the original data, while FIG. 6A plots the ratio γ against the specimen number listed in Table 7. Impressively, all negative specimens were accurately predicted with a 100% success rate. Among the positive specimens, all were correctly identified, except for Positive-3. This specimen has n₋ and n₊ values of 15 and 6, respectively, with γ (=0.71) slightly surpassing 0.7, indicating that Positive-3 was incorrectly predicted as negative. This discrepancy might be attributed to a positive specimen with a high Ct value (low viral concentration). By expanding the training spectral set and adjusting the γ threshold value (say 0.75), improved prediction performance can be expected. In total, the constructed RNN model achieved an overall classification accuracy of 99.04%, with a 98.28% accuracy for positive specimens and a perfect 100% accuracy for negative specimens. Subsequently, the RNN regression model used the averaged 21 spectra per positive specimen for Ct value prediction, as shown in FIG. 6B. The prediction plots show an R² of approximately 0.94-0.9 and RMSEs around 1.1-1.3, demonstrating commendable quantification performance. Compared to Figure D, FIG. 6B shows a relatively better result because the spectra used for the regression in blind test are the average of the 21 measured spectra.

TABLE 7
Blind test result for 104 deidentified HNS specimens.

| | Actual | Predicted label | | | Diagnosis |
|---|---|---|---|---|---|
| Number | label | n₋ | n₊ | γ | result |
| 1 | negative-1 | 21 | 0 | 1.00 | negative |
| 2 | negative-2 | 21 | 0 | 1.00 | negative |
| 3 | negative-3 | 21 | 0 | 1.00 | negative |
| 4 | negative-4 | 21 | 0 | 1.00 | negative |
| 5 | negative-5 | 21 | 0 | 1.00 | negative |
| 6 | negative-6 | 21 | 0 | 1.00 | negative |
| 7 | negative-7 | 21 | 0 | 1.00 | negative |
| 8 | negative-8 | 21 | 0 | 1.00 | negative |
| 9 | negative-9 | 21 | 0 | 1.00 | negative |
| 10 | negative-10 | 21 | 0 | 1.00 | negative |
| 11 | negative-11 | 21 | 0 | 1.00 | negative |
| 12 | negative-12 | 21 | 0 | 1.00 | negative |
| 13 | negative-13 | 21 | 0 | 1.00 | negative |
| 14 | negative-14 | 21 | 0 | 1.00 | negative |
| 15 | negative-15 | 21 | 0 | 1.00 | negative |
| 16 | negative-16 | 21 | 0 | 1.00 | negative |
| 17 | negative-17 | 21 | 0 | 1.00 | negative |
| 18 | negative-18 | 19 | 2 | 0.90 | negative |
| 19 | negative-19 | 21 | 0 | 1.00 | negative |
| 20 | negative-20 | 21 | 0 | 1.00 | negative |
| 21 | negative-21 | 19 | 2 | 0.90 | negative |
| 22 | negative-22 | 21 | 0 | 1.00 | negative |
| 23 | negative-23 | 21 | 0 | 1.00 | negative |
| 24 | negative-24 | 20 | 1 | 0.95 | negative |
| 25 | negative-25 | 21 | 0 | 1.00 | negative |
| 26 | negative-26 | 21 | 0 | 1.00 | negative |
| 27 | negative-27 | 21 | 0 | 1.00 | negative |
| 28 | negative-28 | 21 | 0 | 1.00 | negative |
| 29 | negative-29 | 17 | 4 | 0.81 | negative |
| 30 | negative-30 | 21 | 0 | 1.00 | negative |
| 31 | negative-31 | 21 | 0 | 1.00 | negative |
| 32 | negative-32 | 21 | 0 | 1.00 | negative |
| 33 | negative-33 | 17 | 4 | 0.81 | negative |
| 34 | negative-34 | 17 | 4 | 0.81 | negative |
| 35 | negative-35 | 21 | 0 | 1.00 | negative |
| 36 | negative-36 | 21 | 0 | 1.00 | negative |
| 37 | negative-37 | 21 | 0 | 1.00 | negative |
| 38 | negative-38 | 21 | 0 | 1.00 | negative |
| 39 | negative-39 | 17 | 4 | 0.81 | negative |
| 40 | negative-40 | 21 | 0 | 1.00 | negative |
| 41 | negative-41 | 19 | 2 | 0.90 | negative |
| 42 | negative-42 | 18 | 3 | 0.86 | negative |
| 43 | negative-43 | 21 | 0 | 1.00 | negative |
| 44 | negative-44 | 21 | 0 | 1.00 | negative |
| 45 | negative-45 | 19 | 2 | 0.90 | negative |
| 46 | negative-46 | 21 | 0 | 1.00 | negative |
| 47 | positive-1 | 0 | 21 | 0.00 | positive |
| 48 | positive-2 | 0 | 21 | 0.00 | positive |
| 49 | positive-3 | 15 | 6 | 0.71 | negative |
| 50 | positive-4 | 0 | 21 | 0.00 | positive |
| 51 | positive-5 | 0 | 21 | 0.00 | positive |
| 52 | positive-6 | 0 | 21 | 0.00 | positive |
| 53 | positive-7 | 0 | 21 | 0.00 | positive |
| 54 | positive-8 | 0 | 21 | 0.00 | positive |
| 55 | positive-9 | 0 | 21 | 0.00 | positive |
| 56 | positive-10 | 2 | 19 | 0.10 | positive |
| 57 | positive-11 | 0 | 21 | 0.00 | positive |
| 58 | positive-12 | 0 | 21 | 0.00 | positive |
| 59 | positive-13 | 0 | 21 | 0.00 | positive |
| 60 | positive-14 | 0 | 21 | 0.00 | positive |
| 61 | positive-15 | 11 | 10 | 0.52 | positive |
| 62 | positive-16 | 0 | 21 | 0.00 | positive |
| 63 | positive-17 | 0 | 21 | 0.00 | positive |
| 64 | positive-18 | 0 | 21 | 0.00 | positive |
| 65 | positive-19 | 0 | 21 | 0.00 | positive |
| 66 | positive-20 | 0 | 21 | 0.00 | positive |
| 67 | positive-21 | 0 | 21 | 0.00 | positive |
| 68 | positive-22 | 0 | 21 | 0.00 | positive |
| 69 | positive-23 | 0 | 21 | 0.00 | positive |
| 70 | positive-24 | 3 | 18 | 0.14 | positive |
| 71 | positive-25 | 5 | 16 | 0.24 | positive |
| 72 | positive-26 | 0 | 21 | 0.00 | positive |
| 73 | positive-27 | 0 | 21 | 0.00 | positive |
| 74 | positive-28 | 0 | 21 | 0.00 | positive |
| 75 | positive-29 | 11 | 10 | 0.52 | positive |
| 76 | positive-30 | 0 | 21 | 0.00 | positive |
| 77 | positive-31 | 0 | 21 | 0.00 | positive |
| 78 | positive-32 | 0 | 21 | 0.00 | positive |
| 79 | positive-33 | 0 | 21 | 0.00 | positive |
| 80 | positive-34 | 5 | 16 | 0.24 | positive |
| 81 | positive-35 | 3 | 18 | 0.14 | positive |
| 82 | positive-36 | 0 | 21 | 0.00 | positive |
| 83 | positive-37 | 0 | 21 | 0.00 | positive |
| 84 | positive-38 | 0 | 21 | 0.00 | positive |
| 85 | positive-39 | 0 | 21 | 0.00 | positive |
| 86 | positive-40 | 7 | 14 | 0.33 | positive |
| 87 | positive-41 | 0 | 21 | 0.00 | positive |
| 88 | positive-42 | 0 | 21 | 0.00 | positive |
| 89 | positive-43 | 0 | 21 | 0.00 | positive |
| 90 | positive-44 | 0 | 21 | 0.00 | positive |
| 91 | positive-45 | 8 | 13 | 0.38 | positive |
| 92 | positive-46 | 0 | 21 | 0.00 | positive |
| 93 | positive-47 | 0 | 21 | 0.00 | positive |
| 94 | positive-48 | 0 | 21 | 0.00 | positive |
| 95 | positive-49 | 0 | 21 | 0.00 | positive |
| 96 | positive-50 | 0 | 21 | 0.00 | positive |
| 97 | positive-51 | 0 | 21 | 0.00 | positive |
| 98 | positive-52 | 0 | 21 | 0.00 | positive |
| 99 | positive-53 | 0 | 21 | 0.00 | positive |
| 100 | positive-54 | 0 | 21 | 0.00 | positive |
| 101 | positive-55 | 0 | 21 | 0.00 | positive |
| 102 | positive-56 | 0 | 21 | 0.00 | positive |
| 103 | positive-57 | 0 | 21 | 0.00 | positive |
| 104 | positive-58 | 0 | 21 | 0.00 | positive |

Clearly, our SERS-DLA direct detection strategy has achieved comparable results to RT-PCR. In comparison to other reported methods, our approach demonstrates superior accuracies. For example, rapid antigen detection achieves ˜66% accuracy [41], breath detection via gas chromatography-mass spectrometry reaches 91.2% for positive and 99.3% for negative specimens [40, 42], nucleic acid tests by various commercial products attain ˜95% accuracy [43], and dipstick detection using a Palm Germ-Radar achieves 97.2% accuracy [44]. Remarkably, the entire detection process takes just 15 minutes. These findings indicate the potential of the AgNR@SiO₂ array SERS-DLA as a rapid and promising point-of-care COVID-19 diagnostic platform.

### CONCLUSIONS

In summary, a rapid detection and quantification of SARS-CoV-2 infection directly from HNS specimens using SERS combining with DLAs has been developed, which can yield results comparable to RT-PCR technique. The entire process, from HNS specimen deactivation, SERS specimen preparation and drying, SERS measurements from AgNR@SiO₂ array substrate, and classification via RNN, takes less than 15 min. With an optimized trained RNN model, the classification accuracy of SERS spectra can be as good as 97.1% for positive specimens and 100% for negative specimens. By correlating DL-selected feature importance with the signature ranges of known biomolecules and chemical functional groups, the RNN model effectively recognizes the SERS peaks of proteins, lipids, and other vital functional groups presenting in positive specimens. Furthermore, the RNN regression model enables accurately prediction of RT-PCR Ct values of HNS specimens. Both classification and quantification of HNS specimens can be achieved based on inherent SERS spectral differences within viral components. Finally, for blind SARS-CoV-2 diagnosis, 99.04% accuracy is achieved with good quantification performance. These findings suggest that the use of SERS-DLA to directly detect and quantify SARS-CoV-2 infection from inactivated HNS specimens is a straightforward and cost-effective substitute for RT-PCR and can serve as a reliable and rapid point-of-care platform for direct COVID-19 diagnostics.

### Additional Details

SERS Substrate Description. According to previous study, the AgNR array has been demonstrated to possess a high SERS enhancement factor (up to 109), a good reproducibility (˜10% relative variation), and a large uniformity. Furthermore, when the AgNR array is coated with a uniform and ultra-thin silica layer by the hydrolysis of tetraethylorthosilicate, the issues of surface contamination, stability, and biocompatibility can be resolved and the AgNR@SiO₂ substrate can serve as an excellent SERS substrate for direct virus detection, as demonstrated by our recent publication.³ FIG. 7 shows a representative SEM image of AgNR@SiO₂ array. The surface is composed of tilted nanorods with a wide range of morphologies, such as corrugations, bifurcations, protrusions, as well as random irregularities. The nanorod density is 12±1 rods/μm², the average diameter of the AgNR@SiO₂ is 100 nm, and the length is approximated as ˜1000 nm with a tilted angle of 77±1°.

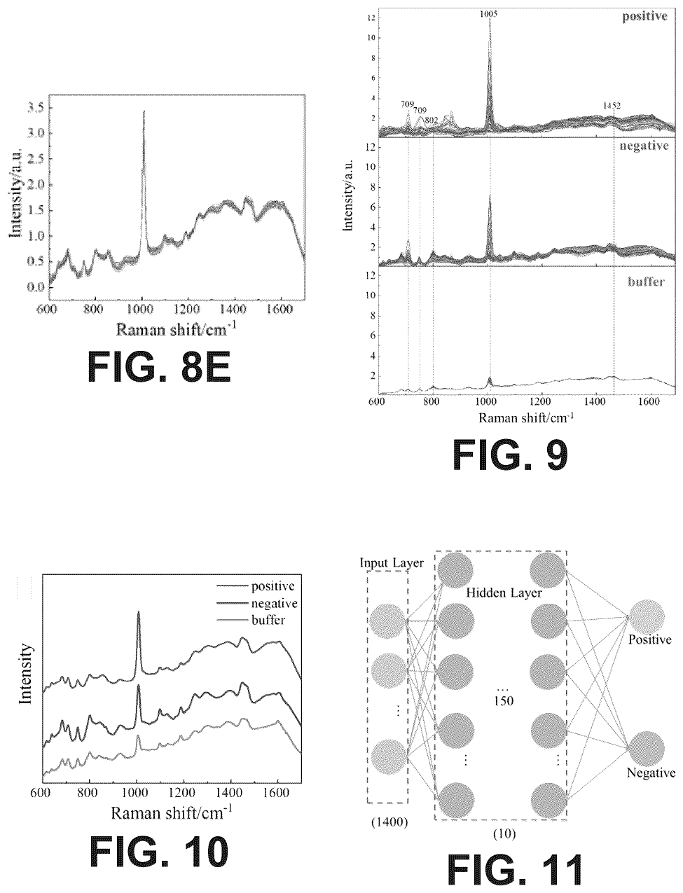

SERS Spectrum Preprocessing. According to the baseline of SERS spectra from Tec5 Raman instrument, a Gaussian-Lorentzian baseline correction method was developed to obtain more unform SERS spectra for deep learning process.^{3,28} To demonstrate this method, a typical raw spectrum (FIG. 8A) was used as an example. First, the featureless spectral ranges of 300-400 cm⁻¹ and 1800-2500 cm⁻¹ for each spectrum can be fitted by the function,

$${I}_{S E R S} ( Δ v ) = A {e}^{- \frac{{( Δ v - {v}_{g} )}^{2}}{2 {σ}_{g}^{2}}} + \frac{2 L {σ}_{l}}{4 {π ( Δ v - {v}_{l} )}^{2} + {σ}_{l}^{2}} + {I}_{0} , \qquad ( S 1 )$$

where A is the amplitude of a Gaussian function, v_{g} is the center of the Gaussian peak, σ_{g} is the standard deviation of the Gaussian function, L is the area of a Lorentzian function, vₗ is the center of the Lorentzian peak, σₗ relates to the width of the Lorentzian peak, I₀ is the “ground” level of the SERS spectrum. The red dashed curve in FIG. 8B shows the fitting result. Then the baseline corrected spectrum is obtained by subtracting the above fitted curve using the original spectrum, see FIG. 8C. Finally, the mean value of each baseline corrected spectrum is calculated, and the final spectrum is normalized by this mean value, see FIG. 8D. Typical SERS spectra of one positive specimen after pre-processing are shown in FIG. 8E (total 20 spectra).

Detailed Information on the Classification Models. SVM and RF analyses were performed in MATLAB and GridSearch was used to optimize the parameters. Penalty coefficient and σ(tuning the speed of training and prediction) in the kernel function for SVM were 103 and 39, respectively. The optimal parameter n_estimators of RF was 927.

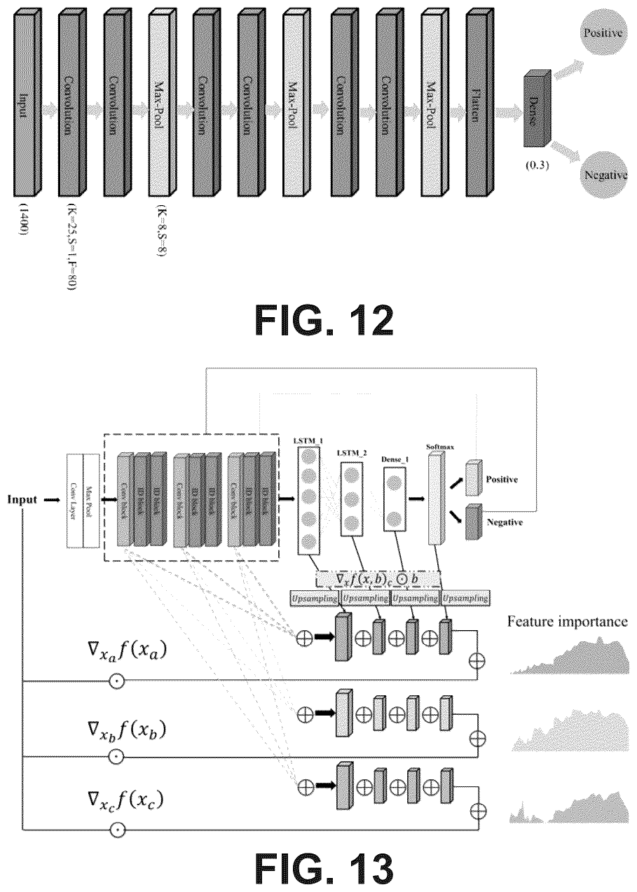

The back-propagation (BP) neural network model is shown in FIG. 13. The BP model included ten hidden layers and 150 units for each layer, and a cross-entropy loss function was used in the training. Adam optimization algorithm with a learning rate of 0.002 was introduced to solve the problem of gradient descent of the loss function. Regularization terms were added to improve the generalization ability and avoid overfitting for the training. The intensity value of each band corresponded to an input. After the calculation of the neurons of the hidden layer, the corresponding Positive or Negative output value was obtained by fitting. Whereas for SVMs, which were trained using the idea of classification intervals, it relied on preprocessing of the data, i.e., expressing the original patterns in a higher dimensional space. The raw data belonging to the two classes were separated by a hyperplane through an appropriate nonlinear mapping to a sufficiently high dimensionality.

For the convolutional neural network (CNN) model as shown in FIG. 14, it had six convolutional layers, three pooling layers, and one fully connected layer with 1400 units. The size of the convolutional kernel was 3×1, 3×1, 5×1, 5×1, 7×1, and 7×1, respectively. The number of filters was 8, 8, 32, 32, 64, and 64, respectively. Cross-entropy loss function was used in the training. The learning rate of the Adam optimization algorithm was 0.002. The input for the model was the intensity value corresponding to each band of the spectrum, and the Input layer was followed by two convolution layers and a Max-Pool layer, repeated three times, followed by the Flatten layer, and finally a Dense layer, judging the sample as positive or negative.

The Ct Value and Other Regression Models. The cycle threshold (Ct) is often used to quantify the amount of SARS-CoV-2 RNA in a specimen after the polymerase chain reaction (PCR) test. The Ct value is the number of cycles needed for the fluorescent signal to cross a certain threshold limit, indicating the presence of the SARS-CoV-2 RNA sequence in the specimen. That is, after Ct circles, the concentration of RNA reaches to certain amount (N_{c}) to obtain the fluorescent signal above the threshold. Then, we can have

$${N}_{c} = {N}_{0} \times {2}^{C t} , \qquad ( S 4 )$$

where N₀ is the initial concentration of SARS-CoV-2 RNA. Thus,

$${N}_{0} = \frac{{N}_{c}}{{2}^{Ct}} , \\ i . e . , \qquad ( S 5 )$$

$${N}_{0} ∝ {2}^{- C t} , Or , \qquad ( S6 )$$

$$In {N}_{0} = - k \times ln 2 \times Ct . \qquad ( S7 )$$

Therefore, the Ct value is inversely proportional to ln N₀ of SARS-CoV-2 RNA in the specimen, meaning that a lower Ct value indicates a higher concentration of SARS-CoV-2 RNA in the specimen.

Typically, PCR amplification is performed over a range of cycles, commonly between 35 and 45 cycles, the RNA copies can be amplified to 2³⁵=3.4×10¹⁰. To compare the limit of detection (LOD) of our method with that of PCR, factors such as specimen dilution, sample volume, and laser beam size need to be taken into consideration. In brief, a 30 μL aliquot of the collected HNS specimen was mixed 1:1 (v:v) with the inactivation buffer. Following this, 10 μL of the mixture was diluted by 300 μL pure water. Then 20 μL of above specimen lysate was dispensed onto a AgNR@SiO₂ well for SERS measurements. The diameter of the well is 4 mm, and the laser beam diameter is approximately 100 μm. And 21 SERS spectra were collected for each specimen. In comparison to PCR, the relative viral load for the SERS measurement can be calculated as follows,

$$\frac{10}{60} \times \frac{20}{310} \times \frac{π \times 5 {0}^{2} \times 21}{π \times 200 {0}^{2}} = 1.411 \times 1 {0}^{- 4} . 1.411 \times 1 {0}^{- 4} \times {n}_{o} \times {2}^{n} \times 20 μ L = {n}_{o} \times {2}^{40} \times 60 μ L n = 54.376$$

For random forest regression and support vector regression (SVR), the optimal parameters are obtained using grid search in the MATLAB environment, with n_estimators of 212 and kernel function of radial basis function (rbf), penalty factor of 163, and a of 0.1 for SVR.

The BP regression model has eight hidden layers, each including 60 neurons, with L1 loss used as the loss function, Adam as the optimizer, and a learning rate of 0.001. The CNN regression model including two convolutional layers, one pooling layer, and three fully connected layers. The kernel sizes for the convolutional layers are 5×1 and 5×1, the kernel size for the pooling layer is 2×1 with a stride of 2, and the fully connected layers have sizes of 400, 120, and 84, with L1 loss used as the loss function, Adam as the optimizer, and a learning rate of 0.002.

### REFERENCES FOR EXAMPLE 1

1. Kneipp, K., et al., Single molecule detection using surface-enhanced Raman scattering (SERS). Physical review letters, 1997. 78(9): p. 1667.
2. Nie, S. and S. R. Emory, Probing Single Molecules and Single Nanoparticles by Surface-Enhanced Raman Scattering. Science, 1997. 275(5303): p. 1102-1106.
3. Yang, Y., et al., Rapid and quantitative detection of respiratory viruses using surface-enhanced Raman spectroscopy and machine learning. Biosensors and Bioelectronics, 2022. 217: p. 114721.
4. Paria, D., et al., Label-Free Spectroscopic SARS-CoV-2 Detection on Versatile Nanoimprinted Substrates. Nano Letters, 2022. 22(9): p. 3620-3627.
5. Ye, J., et al., Accurate virus identification with interpretable Raman signatures by machine learning. Proceedings of the National Academy of Sciences, 2022. 119(23): p. e2118836119.
6. Moitra, P., et al., Probing the mutation independent interaction of DNA probes with SARS-CoV-2 variants through a combination of surface-enhanced Raman scattering and machine learning. Biosensors and Bioelectronics, 2022. 208: p. 114200.
7. Yang, Y., et al., Rapid Detection of SARS-CoV-2 RNA in Human Nasopharyngeal Specimens Using Surface-Enhanced Raman Spectroscopy and Deep Learning Algorithms. ACS Sensors, 2023. 8(1): p. 297-307.
8. Yang, Y., et al., Human ACE2-Functionalized Gold “Virus-Trap” Nanostructures for Accurate Capture of SARS-CoV-2 and Single-Virus SERS Detection. Nano-Micro Letters, 2021. 13(1): p. 109.
9. Zhang, M., et al., Ultrasensitive detection of SARS-CoV-2 spike protein in untreated saliva using SERS-based biosensor. Biosensors and Bioelectronics, 2021. 190: p. 113421.
10. Zhang, J., et al., Non-enzymatic signal amplification-powered point-of-care SERS sensor for rapid and ultra-sensitive assay of SARS-CoV-2 RNA. Biosensors and Bioelectronics, 2022. 212: p. 114379.
11. Carlomagno, C., et al., COVID-19 salivary Raman fingerprint: innovative approach for the detection of current and past SARS-CoV-2 infections. Scientific Reports, 2021. 11(1): p. 4943.
12. Auner, G. W., et al., Counter-propagating Gaussian beam enhanced Raman spectroscopy for rapid reagentless detection of respiratory pathogens in nasal swab samples. Biosensors and Bioelectronics: X, 2022. 12: p. 100230.
13. Karunakaran, V., et al., A non-invasive ultrasensitive diagnostic approach for COVID-19 infection using salivary label-free SERS fingerprinting and artificial intelligence. Journal of Photochemistry and Photobiology B: Biology, 2022. 234: p. 112545.
14. Huang, J., et al., On-Site Detection of SARS-CoV-2 Antigen by Deep Learning-Based Surface-Enhanced Raman Spectroscopy and Its Biochemical Foundations. Analytical Chemistry, 2021. 93(26): p. 9174-9182.
15. Peng, Y., et al., Identifying infectiousness of SARS-CoV-2 by ultra-sensitive SnS2 SERS biosensors with capillary effect. Matter, 2022. 5(2): p. 694-709.
16. Zhang, Z., et al., Rapid detection of viruses: Based on silver nanoparticles modified with bromine ions and acetonitrile. Chemical Engineering Journal, 2022. 438: p. 135589.
17. Hyun Lee, S., et al., 3D interior hotspots embedded with viral lysates for rapid and label-free identification of infectious diseases. Chemical Engineering Journal, 2023. 454: p. 140066.
18. Ansah, I. B., et al., In-situ fabrication of 3D interior hotspots templated with a protein@Au core-shell structure for label-free and on-site SERS detection of viral diseases. Biosensors and Bioelectronics, 2023. 220: p. 114930.
19. Hwang, C. S. H., et al., Highly Adsorptive Au-TiO2 Nanocomposites for the SERS Face Mask Allow the Machine-Learning-Based Quantitative Assay of SARS-CoV-2 in Artificial Breath Aerosols. ACS Applied Materials & Interfaces, 2022. 14(49): p. 54550-54557.
20. Ke, Z., et al., Structures and distributions of SARS-CoV-2 spike proteins on intact virions. Nature, 2020. 588(7838): p. 498-502.
21. Ge, X.-Y., et al., Isolation and characterization of a bat SARS-like coronavirus that uses the ACE2 receptor. Nature, 2013. 503(7477): p. 535-538.
22. Li, F., Structure, Function, and Evolution of Coronavirus Spike Proteins. Annual Review of Virology, 2016. 3(1): p. 237-261.
23. Liu, Y. J., H. Y. Chu, and Y. P. Zhao, Silver Nanorod Array Substrates Fabricated by Oblique Angle Deposition: Morphological, Optical, and SERS Characterizations. The Journal of Physical Chemistry C, 2010. 114(18): p. 8176-8183.
24. Song, C., et al., Ag—SiO2 Core-Shell Nanorod Arrays: Morphological, Optical, SERS, and Wetting Properties. Langmuir, 2012. 28(2): p. 1488-1495.
25. Liu, Y. J. and Y. P. Zhao, Simple model for surface-enhanced Raman scattering from tilted silver nanorod array substrates. Physical Review B, 2008. 78(7): p. 075436.
26. Liu, Y.-J., et al., Surface enhanced Raman scattering from an Ag nanorod array substrate: the site dependent enhancement and layer absorbance effect. The Journal of Physical Chemistry C, 2009. 113(22): p. 9664-9669.
27. Abell, J. L., et al., Fabrication and characterization of a multiwell array SERS chip with biological applications. Biosensors and Bioelectronics, 2009. 24(12): p. 3663-3670.
28. Yang, Y., et al., Differentiation and classification of bacterial endotoxins based on surface enhanced Raman scattering and advanced machine learning. Nanoscale, 2022. 14(24): p. 8806-8817.
29. Pastori no, B., et al., Evaluation of Chemical Protocols for Inactivating SARS-CoV-2 Infectious Samples. Viruses, 2020. 12(6).
30. Privalov, P. L., Cold Denaturation of Protein. Critical Reviews in Biochemistry and Molecular Biology, 1990. 25(4): p. 281-306.
31. Vollenbroich, D., et al., Mechanism of inactivation of enveloped viruses by the biosurfactant surfactin fromBacillus subtilis. 1997. 25(3): p. 289-297.
32. LeCun, Y., Y. Bengio, and G. Hinton, Deep learning. Nature, 2015. 521(7553): p. 436-444.
33. Srinivas, S. and F. Fleuret, Full-gradient representation for neural network visualization, in Proceedings of the 33rd International Conference on Neural Information Processing Systems. 2019, Curran Associates Inc. p. Article 371.
34. Rygula, A., et al., Raman spectroscopy of proteins: a review. Journal of Raman Spectroscopy, 2013. 44(8): p. 1061-1076.
35. Williams, R. W., Protein secondary structure analysis using Raman amide I and amide III spectra, in Methods in Enzymology. 1986, Academic Press. p. 311-331.
36. Godet, M., et al., TGEV corona virus ORF4 encodes a membrane protein that is incorporated into virions. Virology, 1992. 188(2): p. 666-675.
37. Shi, Y., et al., Thiol-based chemical probes exhibit antiviral activity against SARS-CoV-2 via allosteric disulfide disruption in the spike glycoprotein. Proceedings of the National Academy of Sciences, 2022. 119(6): p. e2120419119.
38. Heid, C. A., et al., Real time quantitative PCR. 1996. 6(10): p. 986-994.
39. Vasilescu, A., et al., Exhaled breath biomarker sensing. Biosensors and Bioelectronics, 2021. 182: p. 113193.
40. Arnold, C., Diagnostics to take your breath away. Nature Biotechnology, 2022. 40(7): p. 990-993.
41. Lisboa Bastos, M., et al., Diagnostic accuracy of serological tests for covid-19: systematic review and meta-analysis. BMJ, 2020. 370: p. m2516.
42. Rubin, R., First Breathalyzer Test to Diagnose COVID-19. JAMA, 2022. 327(19): p. 1860-1860.
43. Afzal, A., Molecular diagnostic technologies for COVID-19: Limitations and challenges. Journal of Advanced Research, 2020. 26: p. 149-159.
44. Shim, J.-E., et al., Single-Nanoparticle-Based Digital SERS Sensing Platform for the Accurate Quantitative Detection of SARS-CoV-2. ACS Applied Materials & Interfaces, 2022.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. The term “about” can include traditional rounding according to significant figures of the numerical value. In addition, the phrase “about ‘x’ to ‘y’” includes “about ‘x’ to about ‘y’”.

Many variations and modifications may be made to the above-described aspects. All such modifications and variations are intended to be included herein within the scope of this disclosure and protected by the following claims.

What is claimed:

1. A method for detecting the presence of a biological agent comprising:
disposing a sample onto a surface enhanced Raman spectroscopy (SERS) detecting module, wherein the SERS detecting module comprises a substrate having an array of nanorods on a surface of the substrate, wherein the tilt angle (p) between an individual nanorod and the surface is about 0° to about 90°;
measuring at least one SERS spectrum; and
providing the SERS spectrum to a first recurrent neural network (RNN) model trained to detect the presence or absence of a biological agent in the sample.

2. The method of claim 1, wherein the first RNN model comprises one or more sets of layers, wherein the one or more sets of layers includes:
at least one convolutional layer;
at least one pool layer;
three consecutive blocks comprising a convolutional block, a first identity block, and a second identity block;
at least two recurrent layers; and
at least one fully connected layer.

3. The method of claim 2, wherein the convolutional block comprises a convolutional layer, a batch normalization step, a corrected linear transform step, and a pool layer.

4. The method of claim 2, wherein at least one of the first identity block and the second identity block comprises a convolutional layer, a batch normalization step, a corrected linear transform step, and a pool layer.

5. The method of claim 2, wherein the set of recurrent layers comprises a set of two long short-term memory layers.

6. The method of claim 1, wherein the biological agent is present in the sample, further including providing the SERS spectrum to a second RNN model trained to quantify the amount of biological agent present.

7. The method of claim 6, wherein the second RNN model comprises one or more sets of layers, wherein the one or more sets of layers includes:
at least two recurrent layers,
at least two dropout layers, and
at least three fully connected layers.

8. The method of claim 7, wherein the set of recurrent layers comprises a set of two long short-term memory layers.

9. The method of claim 1, wherein the nanorods are selected from one of the following materials: a metal, a metal oxide, a metal nitride, a metal oxynitride, a polymer, a multicomponent material, and a combination thereof.

10. The method of claim 9, wherein the material is selected from one of the following: silver, nickel, aluminum, silicon, gold, platinum, palladium, titanium, cobalt, copper, zinc, oxides of each, nitrides of each, oxynitrides of each, carbides of each, and a combination thereof.

11. The method of claim 1, wherein the substrate comprises silver nanorods coated with SiO₂.

12. The method of claim 1, wherein the method of detecting takes about 15 minutes or less.

13. The method of claim 1, wherein the biological agent is a type of virus.

14. The method of claim 13, wherein the virus is a member of the subfamily Orthocoronavirinae.

15. The method of claim 14, wherein the virus is SARS-CoV-2 or a variant thereof.

16. The method of claim 1, wherein the sample is selected from blood, saliva, tears, phlegm, sweat, urine, plasma, lymph, spinal fluid, cells, microorganisms, aqueous dilutions thereof, and a combination thereof.

17. The method of claim 1, wherein the sample is obtained from human nasopharyngeal swabs.

18. A system for detecting the presence of a biological agent comprising:
a SERS detecting module having the characteristic of being able to receive a sample, wherein the SERS detecting module comprises a substrate having an array of nanorods on a surface of the substrate, wherein the tilt angle (p) between an individual nanorod and the surface is about 0° to about 90°;
a light source that is directed towards the substrate;
a SERS detection system to measure at least one surface enhanced Raman spectroscopy (SERS) spectrum; and
an analysis system configured to receive the SERS spectrum, wherein the analysis system includes a first recurrent neural network (RNN) model trained to detect the presence or absence of a biological agent in the sample and a second RNN model trained to quantify the amount of biological agent present.

19. The system of claim 18, wherein the first RNN model comprises one or more sets of layers, wherein the one or more sets of layers includes:
at least one convolutional layer;
at least one pool layer;
three consecutive blocks comprising a convolutional block, a first identity block, and a second identity block;
at least two recurrent layers; and
at least one fully connected layer.

20. The method of claim 18, wherein the second RNN model comprises one or more sets of layers, wherein the one or more sets of layers includes:
at least two recurrent layers,
at least two dropout layers, and
at least three fully connected layers.
